(19) United States
(12) Patent Application Publication
(10) Pub. No.: US 2011/0116969 A1
(43) Pub. Date: May 19, 2011

(54) WASTE RECEPTACLE SANITIZING SYSTEM AND METHOD

(76) Inventor: Russell Wallis, Phoenix, AZ (US)

(21) Appl. No.: 12/948,024

(22) Filed: Nov. 17, 2010

Related U.S. Application Data
(60) Provisional application No. 61/262,286, filed on Nov. 18, 2009.

(51) Int. Cl. A61L 2/22

(52) U.S. Cl. 422/28; 422/302

(57) ABSTRACT

A system and method for sanitizing waste receptacles by automatically releasing sanitizing agents in the waste receptacles. A canister containing a sanitizing agent is mounted inside a waste receptacle in order to release its sanitizing agent. Either a mechanical or electromechanical actuator releases at least a portion of the sanitizing agent to control unpleasant odors.

### CROSS-REFERENCE TO RELATED APPLICATIONS

This application claims priority to U.S. Provisional Application Ser. No. 61/262,286 filed on Nov. 18, 2009, and entitled “WASTE RECEPTACLE SANITIZING SYSTEM AND METHOD,” which is herein incorporated by reference.

### FIELD OF INVENTION

This invention generally relates to a system and method for sanitizing waste receptacles such as trash cans by automatically releasing sanitizing agents in waste receptacles.

### BACKGROUND OF THE INVENTION

Garbage is produced in large quantities. A substantial portion of this garbage produces unpleasant odors. Typically garbage collection occurs once a week. Between collection times the garbage and resulting odors continually accumulate. Because it is preferable to locate garbage receptacles in convenient locations (i.e. locations were it is easy to dispose of garbage at any time) these odors are also located in these same locations, becoming highly inconvenient. The waste inside a garbage receptacle quickly produces pungent spoiled odors enveloping the area surrounding the receptacle. Such issues can be especially problematic for many businesses such as restaurants which produce a particularly large amount of odor producing organic garbage. The resulting odors can at a minimum be annoying and possibly turn customers away. Homeowners may face similar problems in dealing with their own garbage, due to placing garbage receptacles near doorways to the house or in a garage.

The primary odor control mechanism is a lid. The lid functions to seal the odors inside of the garbage receptacles. However, the lids must be opened in order to deposit the garbage into the receptacle, allowing the odor to escape. Furthermore, many lids fit poorly or are left open. As such a solution to these and other problems is needed.

### SUMMARY OF THE INVENTION

The present invention comprises in one exemplary embodiment, a waste receptacle disinfecting apparatus comprising: an actuator, wherein the actuator comprises a lever which is configured to be depressed by a waste receptacle lid; a placement device, wherein the placement device comprises a hook configured to attach over the waste receptacle wall; a safety pin; a spray nozzle, wherein the spray nozzle is configured to be triggered by the actuator; a sanitizing agent; and a canister configured to contain the sanitizing agent.

In another exemplary embodiment the present invention comprises, a waste receptacle disinfecting system comprising: an actuator, wherein the actuator comprises a lever which is configured to be depressed by a lid of the waste receptacle; a spray nozzle, wherein the spray nozzle is configured to be triggered by the actuator; a sanitizing agent; a canister configured to contain the sanitizing agent; and a waste receptacle wherein the actuator is integral with the waste receptacle.

In another exemplary embodiment, the present invention also comprises a method for waste receptacle sanitizing comprising: placing a canister on a waste receptacle wall; closing a lid; triggering an actuator; and releasing a sanitizing agent through a spray nozzle, wherein the actuator contacts spray nozzle.

### BRIEF DESCRIPTION OF THE DRAWINGS

FIG. 1 illustrates an exemplary embodiment of a waste receptacle sanitizing apparatus.

FIG. 2 illustrates an exemplary embodiment of a waste receptacle sanitizing apparatus attached to an exemplary waste receptacle.

### DETAILED DESCRIPTION

The detailed description of exemplary embodiments of the invention herein shows the exemplary embodiment by way of illustration, diagrams and various processing steps including the best mode. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in any order and are not limited to the order presented. Furthermore, any reference to singular includes plural embodiments, and any reference to more than one component or step may include a singular embodiment or step. Also, any reference to attached, fixed, connected, supported or the like may include permanent, removable, temporary, partial, full and/or any other possible attachment or support option. Additionally, any reference to without contact (or similar phrases) may also include reduced contact or minimal contact.

Moreover, for the sake of brevity, certain sub-components of individual components and other aspects of the system may not be described in detail herein. It should be noted that many alternative or additional functional relationships, wireless connections or physical couplings may be present in a practical system. Such functional blocks may be realized by any number of components configured to perform specified functions.

The apparatus of the present invention may sanitize waste receptacles to provide a more pleasant environment in and near the waste receptacle. In one embodiment of the present invention, the apparatus releases a sanitizing agent when a lid of the waste receptacle is closed. In other embodiments of the present invention the apparatus releases a sanitizing chemical after the lid has closed.

A sanitizing agent may include but is not limited to deodorants, sanitizers, neutralizers, disinfectants and absorbents. As used herein sanitizing agents refer to any solid, liquid, gel, or gaseous compounded capable of eliminating undesirable odors or odor causing elements from waste receptacles.

As used herein a lid may include and/or refer to but is not limited to any top, hatch, cover, access, closure, or lid used on or in connection with a waste receptacle for enclosing the container.

As used herein a waste receptacle includes but is not limited to any dumpster, container, enclosure, compartment, can, box, bin, structure or device used for or capable of storing waste, garbage, recycling, or refuse to be disposed of. Such waste receptacles can be located indoors or outdoors.

As in various embodiments of previous invention, the apparatus that releases the sanitizing agent is easily accessible. As the sanitizing agent is depleted it may need to be replaced. In one exemplary embodiment of the present invention the apparatus that releases the agent may be located near the opening to the waste receptacle. In various other exemplary embodiments the apparatus may be located to optimally deliver the sanitizing agent to a waste receptacle. For example in large dumpsters the apparatus may be located where the bulk of the waste is originally piled so that it better sanitizes from the first addition of waste to the last addition of waste prior to removal by a waste service. In another example the apparatus may be located on the lid so as to be out of the way as waste is added to the receptacle.

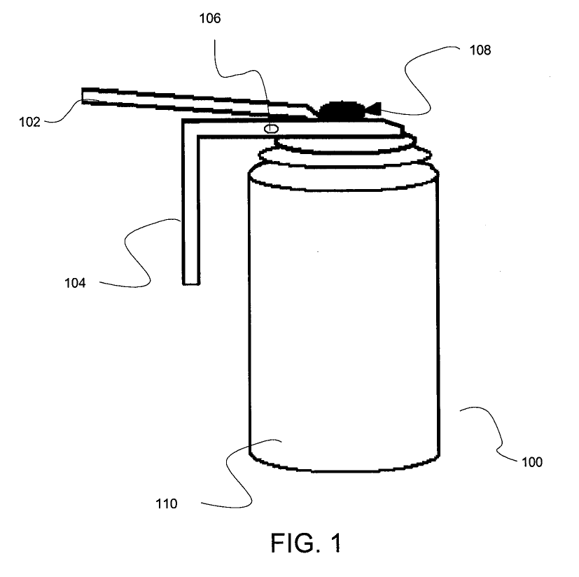

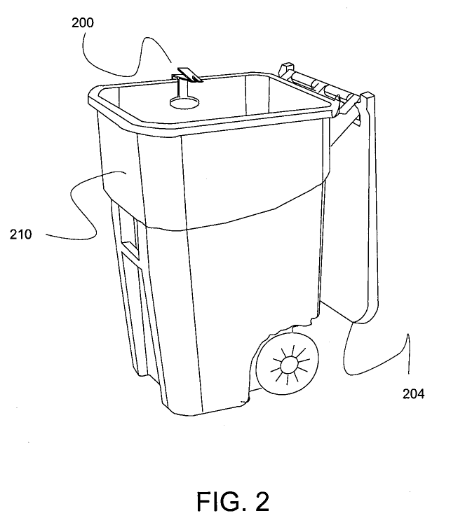

In accordance with one embodiment of present invention, an apparatus comprises an actuator, a placement device, a safety pin, a spray nozzle, and a canister. In reference to FIG. 1, Container 110 may contain a sanitizing agent. The sanitizing agent may be released through nozzle 108. Lever 102 may contact nozzle 108 causing sanitizing agent to be released. Lever 102 may attach to any structure supporting canister 110 such as placement device 104 or to the canister itself. Placement device 104 supports canister 110 and attaches the entire apparatus to a waste receptacle. In reference to FIG. 2, apparatus 200 may mount to the wall of waste receptacle 210. Lid 204 may close, triggering apparatus 200 to release the sanitizing agent.

Actuator 102 may be any mechanical, electro mechanical, or electronic device that allows spray nozzle 108 to be triggered while inside of closed waste receptacle 210. In one exemplary embodiment, sanitizing agent canister 110 may be dispensed due to lid 204 coming into physical contact with actuator 102. In another exemplary embodiment actuator 102 is triggered by a signal from a sensor, timer, or remote. Thus, an individual does not have to be near the waste receptacle to physically dispense sanitizing agent canister 110.

In one exemplary embodiment, shown in FIG. 1, actuator 102 can be a lever, button, post or protrusion that is depressed by waste receptacle lid 204, when it is closed. Still in other various embodiments, actuator 102 can be an electro mechanical devise, which may comprise a button or sensor that can be triggered and send an electronic signal to an electro mechanical device such as a solenoid, motor, moving coil, etc. In such an embodiment, trash receptacle lid 204 would still actuate the button sensor or trigger that sends the electronic signal.

In other various embodiments of the present invention, apparatus 100 can be actuated by a purely electronic signal that is sent from outside of trash receptacle 210 by means of a remote control, timing device, or various other mechanisms that do not rely on any physical contact from the closing of waste receptacle lid 204 or other triggering mechanism.

With continued reference to FIG. 1, apparatus 100 may also comprise placement device 104. Such placement device 104 might comprise any of hooks, clips, clamps, snaps, levers, tabs, magnets, or any mechanism or feature that allows apparatus 100 to be attached to lid 204, rim, walls, or another component and/or feature of waste receptacle 210.

In one exemplary embodiment, placement device 104 may comprise a hook which may fasten apparatus 100 on the wall over the top rim of waste receptacle 210. Furthermore, the hook may be such that it provides a clamping force which engages with lid 204, rim, wall, or other feature such that it is not movable.

In other embodiments, waste receptacle 210 may comprise an attachment component that allows for the attachment of any various accessories such as placement device 104. For example waste receptacle 210 may comprise a bracket for receiving apparatus 100. In one exemplary embodiment tabs from apparatus 100 can clip into waste receptacle 210.

Alternatively, in another exemplary embodiment, placement device 104 can comprise a magnet. A magnetized surface on apparatus 100 may allow apparatus 100 to attach to the side of metal waste receptacle 210. Placement device 104 that prevents apparatus 100 from being easily moved within trash receptacle 210 when lid 204 is closed or when trash receptacle 210 is moved or emptied would be used in a preferred embodiment. However, such a device should also be capable of easy removal or replacement.

In various other exemplary embodiments, apparatus 100 may further comprise a safety interlock 106 or latch that prevents that actuator 102 or electronic system from causing the canister to expel its content prematurely. In one exemplary embodiment, safety interlock 106 is a physical obstruction or other mechanism that prevents actuator 102 from being depressed. Such a physical obstruction might include but is not limited to a pin, lever, switch, lock, and/or latch. Furthermore, the obstruction may be removable or break-away such as plastic tabs. Safety interlock 106 prevents the contents of the can from being expelled before the user desires. Thus, the user can open and close waste receptacle lid 204 without dispelling the contents of apparatus 100 until safety interlock 106 is removed.

Apparatus 100 further comprises spray nozzle 108 and a canister 110. Spray nozzle 108 and canister 110 may contain any commercially available sanitizing agent.

Alternatively, in yet another exemplary embodiment, canister 110 and spray nozzle 108 can be a proprietary design optimally configured to be used with a specific design of waste receptacle 210. Furthermore, canister 110, spray nozzle 108, an actuator 102, placement device 104, and safety interlock 106 may be integrally formed as one part, two parts, or any of a variety separate parts making up apparatus 100. Furthermore apparatus 100 may be formed as a part of the waste receptacle. In one exemplary embodiments, spray nozzle 108 can be designed and configured such that nozzle 108 is integral with actuator 102. Furthermore, canister 110 maybe any of a containment unit that can hold, store, and dispense the sanitizing agent. Container 110 maybe pressurized or unpressurized

In various exemplary embodiments, the apparatus may release a sustained, periodic, or burst of the sanitizing agent. In one such embodiment, actuator 102 and nozzle 108 of apparatus 100 functions to act as a release mechanism which allows apparatus 100 to either create a continuous spray which may deplete the entire contents of canister 100 or an intermittent spray which may allow just a burst of spray that occurs for a finite period of time.

In one exemplary embodiment, the contents of canister 110 can be dispensed each and every time lid 204 of garbage receptacle 210 is opened and closed again, allowing prolong use of the spray. In an alternate exemplary embodiment, the contents of canister 110 can be dispensed in a continuous spray, depleting the contents of the container. The prolonged release mechanism that allows for depletion of the contents of canister 110 allows for a more complete saturating of garbage receptacle 210, allowing for better application of the sanitizing agent.

Because the sanitizing agent is a consumable, it will need replenished regularly. In one exemplary embodiment apparatus 100 itself may be disposable. After the contents of the canister are completely dispersed the apparatus can be disposed of in waste receptacle 210. Subsequently, a new apparatus 100 can be placed in waste receptacle 210. In various other embodiments of the present invention, apparatus 100 can be only partially disposable wherein, only spray nozzle 108 and/or canister 110 may be disposed. Thus only spray nozzle 108 and canister 110 would be replaced. In reference to FIG. 2 apparatus 210 may remain attached to waste receptacle 200 while replacing canister 110 and nozzle 108. In yet another exemplary embodiment only the sanitizing agent may be replaceable. In such an embodiment the canister could be opened and new sanitizing agent could be added.

In one exemplary embodiment, apparatus 100 may also be an integral part of waste receptacle 210. In such a system actuator 102 and safety interlock 106 may be formed into and part of waste receptacle 210. Furthermore, disposable spray nozzle 108 and/or canister 110 can be the replaceable part of the system. Alternatively, spray nozzle 108 can also be an integral part and only canister 110 containing the disinfecting material is exchanged with the system.

In other various embodiments, the entire system can be integral with garbage receptacle 210 (i.e. the apparatus and the waste receptacle may be one contiguous device.) In such an embodiment the sanitizing agent and/or canister may be the only separable components. As such, a system may comprise garbage receptacle 210 wherein, the garbage receptacle has an integrated actuator, a safety mechanism, a canister, a nozzle, and can be refillable with sanitizing agent.

Discussed herein is also a variety of methods for addressing waste receptacles 210 odor issues. In one exemplary embodiment, the method comprises: placing canister 110 on waste receptacle 210 wall; closing lid 204; triggering actuator 102; and releasing canister 110 contents through spray nozzle 108, wherein actuator 102 contacts spray nozzle 108. After canister 110 has released, all of its contents canister 110 and contents can be replaced, throwing away the old canister 110 and replacing it with a new canister 110 contents.

Another exemplary embodiment of a method comprising attaching canister 110 to waste receptacle 210 wall or lid 204; triggering actuator 102 by closing lid 204; causing canister 110 to spray a first spray wherein, closing lid 204 causes actuator 102 to trigger spray nozzle 108 releasing the contents of canister 110 in a finite burst. Each time lid 204 is closed a new burst may be released without having to replace canister 110 between each and every opening and closing of lid 204.

In other various embodiments, actuator 102 system is electro mechanical such that the system can receive a signal from a remote control outside waste receptacle 210. The method comprising placing the system in waste receptacle 210; closing lid 204; and actuating canister 110 mechanism with a remote control from outside lid 204. Alternatively a timer exterior waste receptacle 210 may cause a release of a portion or all of canister 110 contents into waste receptacle 210.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the invention. The scope of the invention is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Furthermore, no element, component, or method step in the present disclosure is intended to be dedicated to the public regardless of whether the element, component, or method step is explicitly recited in the claims. As used herein, the terms “comprises”, “comprising”, or any other variation thereof, are intended to cover a non-exclusive inclusion, such that a process, method, article, or apparatus 100 that comprises a list of elements does not include only those elements but may include other elements not expressly listed or inherent to such process, method, article, or apparatus.

1. A waste environment sanitation and disinfecting device comprising:
a waste receptacle;
a sanitizing agent;
a canister configured to contain the sanitizing agent;
a spray nozzle configured to dispense the sanitizing agent from the canister in response to the spray nozzle being triggered;
an actuator configured to trigger the spray nozzle; and
a placement device configured to attach to the waste receptacle wall;

2. The device of claim 1, wherein the waste receptacle, actuator, and placement device are a single integrated component, wherein the spray nozzle, sanitizing agent and canister are a singular replaceable component.

3. The device of claim 1, wherein the actuator includes a lever which extends from a portion of the placement device adjacent to the spray nozzle to a portion of the placement device that is in contact with the waste receptacle wall, such that the lever is configured to be depressed by a waste receptacle lid in response to closing the lid which causes the lever to contact the spray nozzle and release the sanitizing agent.

4. The device of claim 3, wherein the actuator includes an electro mechanical trigger configured to trigger the spray nozzle.

5. The device of claim 4, wherein the electro mechanical trigger is activated by a signal outside of the waste receptacle.

6. The device of claim 1, wherein the placement device includes a hook configured to provide support to the device by sliding over and hanging from a side wall of the waste receptacle.

7. The device of claim 1 further comprising: a safety interlock configured to prevent the sanitizing agent from dispelling until the safety interlock is removed.

8. A method for waste receptacle sanitizing comprising:
placing a canister on a bracket on a waste receptacle wall;
closing a waste receptacle lid;
triggering an actuator attached to the bracket; and
releasing a sanitizing agent from the canister through a spray nozzle, wherein the actuator contacts the spray nozzle.

9. The method of claim 8, wherein the actuator is configured to be triggered by the closing of the lid.

10. The method of claim 8, wherein the actuator includes a lever which extends from over the spray nozzle to the placement device such that the lever is configured to be depressed by a waste receptacle lid.

11. The method of claim 8, wherein the actuator is an elector mechanical actuator triggered by a signal coming from outside of the waste receptacle.

12. The method of claim 8, wherein the placing of the canister includes attaching a placement device having a hook configured to slide over and hang from a side wall of the waste receptacle.

13. The method of claim 8, further comprising:
removing the canister from the bracket after it is depleted; and
replacing the depleted canister with a new canister full of sanitizing agent.

14. A system for waste environment sanitation, wherein a sanitizing agent is dispensed from a canister into the waste environment, the system comprising: a waste receptacle, an actuator and a placement device, wherein the placement device is configured to support the canister and the placement device is integrated into a wall of the waste receptacle, wherein the actuator attaches to the placement device and is configured to trigger a spray nozzle on the canister.

15. The device of claim 14, wherein the spray nozzle, sanitizing agent and canister are a singular replaceable component.

16. The device of claim 14, wherein the actuator includes a lever which extends from over the spray nozzle to the placement device such that the lever is configured to be depressed by a waste receptacle lid which causes the lever to contact the spray nozzle and release the sanitizing agent.

17. The device of claim 14, wherein the actuator includes an electro mechanical trigger configured to trigger the spray nozzle.

18. The device of claim 17, wherein the electro mechanical trigger is activated by a signal outside of the waste receptacle.

19. The device of claim 14, wherein the placement device includes a hook configured to provide support to the placement device by sliding over a side wall of the waste receptacle.

20. The device of claim 14 further comprising: a safety interlock configured to prevent the sanitizing agent from dispelling until the safety interlock is removed.
